(19) United States
(12) Patent Application Publication
(10) Pub. No.: US 2014/0180156 A1
(43) Pub. Date: Jun. 26, 2014

(54) AEROSOL COLLECTION DEVICES AND METHODS OF USE

(71) Applicants: David Nelson Ku, Decatur, GA (US); Tamera Lee Scholz, Melbourne, FL (US); Sarah Kathryn Ku, Decatur, GA (US); Matthew Taylor Bronikowski, Alpharetta, GA (US)

(72) Inventors: David Nelson Ku, Decatur, GA (US); Tamera Lee Scholz, Melbourne, FL (US); Sarah Kathryn Ku, Decatur, GA (US); Matthew Taylor Bronikowski, Alpharetta, GA (US)

(73) Assignees: GEORGIA TECH RESEARCH CORPORATION, Atlanta, GA (US); MD INNOVATE, Decatur, GA (US)

(21) Appl. No.: 13/885,428

(22) PCT Filed: Nov. 17, 2011
(86) PCT No.: PCT/US2011/061187

§ 371 Date: Nov. 25, 2013

Related U.S. Application Data
(60) Provisional application No. 61/414,708, filed on Nov. 17, 2010.

(51) Int. Cl. A61B 5/08

(52) U.S. Cl. CPC A61B 5/082
USPC 600/532

(57) ABSTRACT

Provided are devices for collecting an aerosol sample from a subject. The devices comprise a conduit that defines an inner space for the passage of exhaled air. An example conduit has a long axis and a first opening for receiving exhaled air from the subject. The devices further comprise a filter for collecting a sample from the exhaled air. Also provided are methods for collecting samples aerosols exhaled from a subject.

### CROSS-REFERENCE TO RELATED APPLICATIONS

This application claims the benefit of U.S. Provisional Patent Application No. 61/414,708, filed Nov. 17, 2010, which is incorporated herein by reference in its entirety.

### TECHNICAL FIELD

The present application relates to devices for collection of aerosol samples from the respiratory system of a subject.

### BACKGROUND

Pneumonia, or an inflammation of the lungs, is a leading cause of morbidity and mortality worldwide. Pneumonia kills more children less than five years of age than AIDS, malaria, and measles combined. In the United States, it has been estimated that each year approximately 1.4 million hospitalizations and 59,000 deaths result from pneumonia.

Pneumonia can be caused by a variety of bacterial and viral pathogens, including Streptococcus pneumoniae, Mycoplasma tuberculosis, Chlamydia pneumonia, Legionella, influenza viruses, respiratory syncytial virus, parainfluenza, adenovirus, rhinovirus, human bocavirus, influenza, Mycoplasma pneumoniae, hantavirus, and cytomegalovirus.

To treat pneumonia appropriately it is important to properly identify the causative pathogen in the lower airways. This can be done by detecting the presence of the pathogen, virus, bacteria or fungus, in the lung, i.e. alveoli and/or bronchioles. It is difficult, however, to obtain a sample from the lung and to determine the etiology of pneumonia.

For example, in the case of one of the leading causes of pneumonia, S. pneumonia, as many as 70% of healthy people carry the bacteria in their upper respiratory system. This makes it impossible to confidently determine if a positive detection for S. Pneumoniae indicates carriage or the cause of pneumonia when using a sputum specimen, which is collected after passing through the upper respiratory tract and mouth and is, therefore, contaminated with upper respiratory tract organisms. More invasive techniques for detecting lung pathogens include laparoscopic alveolar biopsy or lung puncture. These procedures are medically risky, costly and painful.

### SUMMARY

Provided are devices for collecting an aerosol sample from a subject. The devices comprise a conduit that defines an inner space for the passage of exhaled air. The conduit has a long axis and a first opening for receiving exhaled air from the subject. The devices further comprise a filter for collecting a sample from the exhaled air. The filter is in communication with the inner space and is substantially aligned with the first opening. Optionally, the cross-sectional area of the inner space along the conduit taken perpendicular to the long axis is constant between the opening and the filter.

Optionally, a portion of the conduit is configured for placement in the oral cavity of the subject, and wherein the conduit comprises at least one notch spaced from the first opening. The notch is configured to receive a tooth surface of the subject. The distance between the notch and the first opening measured along the body's outer surface is optionally about 1.0 cm or greater. For example, the distance between the notch and the first opening measured along the body's outer surface may be optionally between about 1.0 cm and 5.0 cm. The conduit optionally comprises two notches positioned to receive an upper incisor and a lower incisor of the subject respectfully.

The devices can further comprise a branch segment defining an inner space in communication with inner space of the conduit that extends from the conduit at an angle relative to the long axis of the conduit. The branch segment optionally terminates in a reservoir configured to receive exhaled air. The opening to the branch segment from the conduit optionally has a larger cross-sectional area than a cross sectional area of the conduit taken perpendicular to the long axis. The reservoir optionally has an inner volume less than 300 ml.

The reservoir optionally expands upon filling with exhaled air from the subject. Pressure in the expanded reservoir directs exhaled air along the conduit and through the filter. The exhaled air received in the reservoir increases pressure in the reservoir causing the direction of additional exhaled air along the conduit and through the filter.

The filter may optionally comprise a lung pathogen antigen. Optionally, at least a portion of the antigen is not collected from the exhaled air and at least a portion of the antigen is collected from the exhaled air. Optionally, the devices further comprise a solution containing an antibody specific for the antigen. The conduit optionally further comprises a reservoir configured to receive the solution and channel configured deliver at least a portion of the received solution into contact with the filter. Kits are also provided that include the described devices and antibody specific to a target antigen.

Also provided are methods for collecting sample aerosols exhaled from a subject. An example method of collecting an aerosol sample from a subject comprises providing a conduit that defines an inner space that has a first opening for receiving exhaled air from the subject. Exhaled air from the subject is received into the inner space through the first opening and a filter is positioned in communication with the inner space. The filter is optionally substantially aligned with the first opening. The exhaled air passes through the filter and the filter captures a sample from the exhaled air.

These and other features and advantages will become more readily apparent to those skilled in the art upon consideration of the following detailed description and accompanying drawings, which describe both the preferred and alternative embodiments of the devices, kits and methods.

### BRIEF DESCRIPTION OF THE DRAWINGS

FIG. 1 is a schematic illustration of an example aerosol collection device.

FIG. 2A is a schematic side view of the device of FIG. 1 with portions exploded to show detail.

FIG. 2B is a schematic illustration of a filter for use with the example aerosol collection devices described throughout.

FIG. 2C is a schematic midline longitudinal cross-sectional illustration showing a view of the device of FIGS. 1 and 2A.

FIG. 3A is a schematic illustration showing a view of the device of FIGS. 1 and 2A.

FIG. 3B is a schematic perspective illustration showing a view of the device of FIGS. 1 and 2A.

FIG. 4A is a schematic illustration showing a view of the device of FIGS. 1 and 2A.

FIG. 4B is a schematic illustration showing a view of the device of FIGS. 1 and 2A.

FIG. 5 is a schematic illustration showing a view of the device of FIGS. 1 and 2A.

FIGS. 6A and 6B are schematic illustrations of a cap for use with the example aerosol collection devices described throughout.

FIG. 7A is a schematic illustration of an example aerosol collection device.

FIG. 7B is a schematic illustration of an example aerosol collection device of FIG. 7A with a cap.

FIG. 7C is a perspective schematic illustration of an example aerosol collection device of FIG. 7B.

FIG. 7D is a schematic illustration of a view of the example aerosol collection device of FIGS. 7B and 7C.

FIG. 7E is a schematic illustration of a view of the example aerosol collection device of FIGS. 7B and 7C.

FIG. 8A is a schematic perspective illustration of an example aerosol collection device.

FIG. 8B is a schematic perspective illustration of an example aerosol collection device with portions exploded to show detail.

### DETAILED DESCRIPTION

Provided are devices and methods used to obtain an aerosol sample from a subject's airways. For example, the devices and methods can be used to obtain an aerosol sample exhaled from the airways of a subject having or suspected of having pneumonia. Optionally, the sample comprises material exhaled from the subject's lungs. Optionally, the sample excludes material exhaled from the subject's upper airways or a portion thereof.

The upper airways include the oral cavity and larynx. The lower airways include the trachea, lungs, bronchus, bronchi, bronchioles, alveolar sacs and alveoli. The volume of air in the upper airways is dependent on the size of the subject. For a child, the volume of upper airway air may be as little as 50 ml. For a large adult, the volume may be as much as 300 ml. Typically, an average adult will expel upper airway air in the first 100 to 150 ml of exhaled breath.

The described devices are optionally used to obtain an aerosol sample without sampling some or all of the air exhaled from the upper airways of a subject. For example, the described devices are optionally used to obtain an aerosol sample without sampling some or all of the air exhaled from the oral cavity.

By excluding some or all of the upper airway air during collection, the described devices collect aerosol samples having no, or a reduced quantity of, material from the subject's upper airway air. By excluding, or reducing, materials found in upper airway, the device and methods can be used to provide a sample for more specific, accurate and effective lower airway pathogen detection. Air can be exhaled into the devices by any mechanism. For example, the subject can cough or provide a controlled exhale into the devices. Also exhaled air includes any material found in an exhaled sample from a subject. Thus, exhaled air, as used herein optionally includes pathogens, cells, water droplets or any other material that exits the airway with an exhalation by the subject.

The described devices and methods, therefore, are advantageously used for obtaining an aerosol sample that reduces collected material from at least a portion of the upper airway. The devices and methods are optionally used to improve the detection, diagnosis and treatment of pneumonia. The described devices and methods are optionally used in various other situations such as determining the presence and concentration of alcohol in the blood stream and for detecting and diagnosing disease processes such as lung cancer, coal dust exposure levels or anthrax exposure levels.

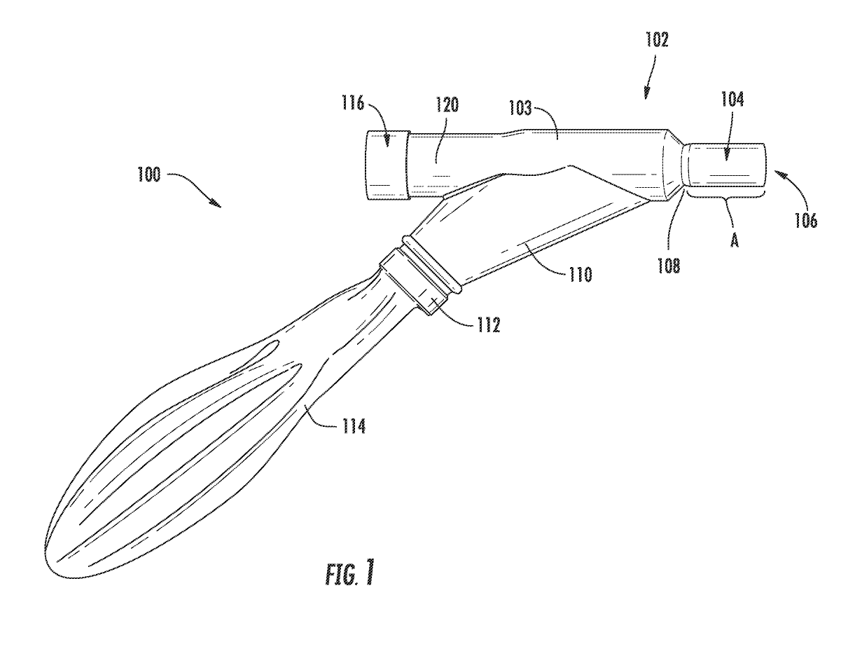

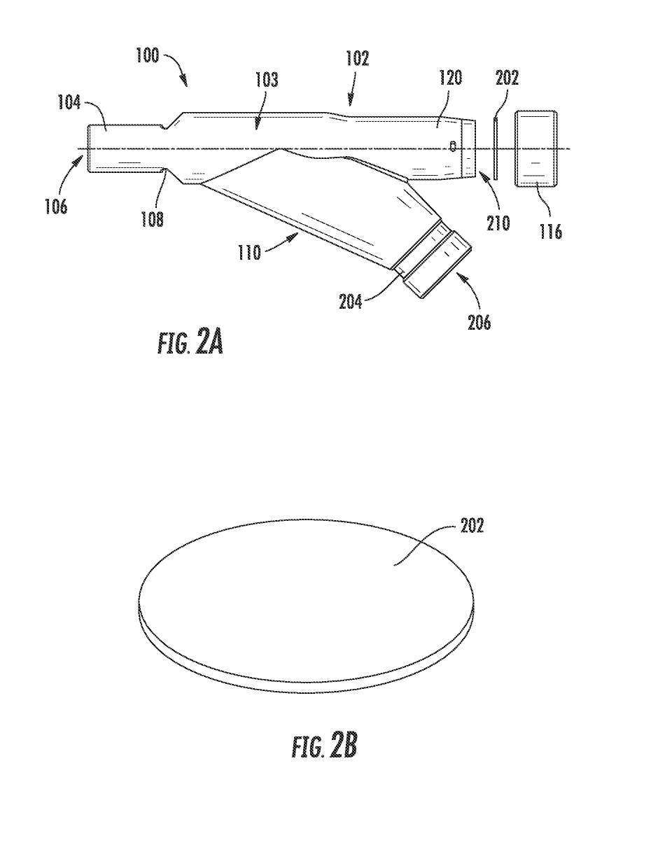

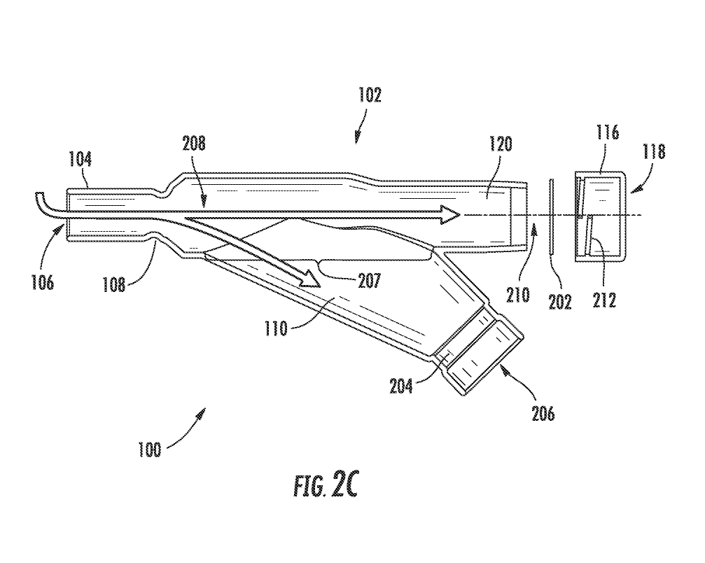

FIG. 1 is a schematic illustration of an example aerosol collection device 100. The device includes a body 102. The body 102 defines an inner space 208 (see FIG. 2C) through which exhaled air, including particles or matter in the air, including water droplets and pathogens can pass. The air exhaled can include air exhaled from the lower airways and the particles or matter can include pathogens causative or complicating for pneumonia.

The body 102 includes conduit 103 comprising a mouthpiece portion 104 having a terminal opening 106. The mouthpiece portion 104 defines a portion of the inner space 208, which is in fluid communication with the opening 106. Air is exhaled by a subject through the opening 106 and it passes though the inner space defined by the mouthpiece portion 104 and the remainder of the body 102. The opening 106 can be any shape. Optionally, the opening 106 is oval, round, rectangular, square or irregular.

The conduit 103 further includes a filter branch portion 120. The body 102 further comprises a reservoir branch portion 110, which branches at an angle from the couduit 103. The filter branch portion 120 has a terminal opening 210 (see FIG. 2C) through which exhaled air may pass. The reservoir branch portion 110 also has a terminal opening 206 through which exhaled air may pass. The conduit 103, including the mouthpiece portion 104 and the filter branch portion 120, directs the passage of exhaled air to a filter in communication with the inner space 208. The reservoir branch portion 110 branches from the conduit at an angle to the long axis of the conduit 103.

The conduit 103 optionally has a linear shape without curvilinear surfaces between the opening 106 and the opening 210. The reservoir branch portion 110 optionally branches at an angle from the conduit 103 between the openings 106 and 210. Optionally, the reservoir branch portion 110 extends from the lower surface of the conduit 103. This change in elevation directs denser liquid material included in exhaled air away from the opening 210. Liquid material has a higher density than aerosolized particles, thus, will tend to travel to the lowest point as fluid moves through the device.

The device 100 is optionally used in a substantially horizontal position with respect to the elevation of the opening 106 and the opening 210. The body 102 is optionally made from polymeric material, metal, glass, and the like.

Optionally, the lumen, or inner space of the conduit 103 extending between the openings 106 and 210 is substantially linear so as to form a cylindrical shape. The circumferential shape or cross-section taken perpendicular to the long axis of the conduit, however, of the inner space of the conduit 103 can vary. The cross-sectional shape is optionally oblong, square, rectangular, triangular, or any other shape.

Similar to the mouthpiece portion 104, the filter branch portion 120, and reservoir branch portion 110 each define portions of the body's inner space. Therefore, air exhaled by a subject through the opening 106 passes through the inner space defined by the mouthpiece portion 104, the reservoir branch portion 110 and the filter branch portion 120.

The inner space 208 defined by the body portions are in fluid communication, and thus, the body 102 as a whole defines a unified inner space for passage of exhaled air from the opening 106 towards the reservoir branch portion 110 and the filter branch portion 120. Optionally, the opening to the reservoir branch portion 207 from the remainder of the inner space 208 is sized to create a lower resistance pathway for the initial volume of exhalation.

The mouthpiece portion 104 is sized and shaped for positioning within the mouth or the oral cavity of a subject. The mouthpiece portion 104 may include at least one notch 108 into which an occlusal or incisal tooth surface may be positioned. Optionally, the mouthpiece portion 104 includes at least two notches 108 positioned on opposite sides of the mouthpiece portion. These opposed notches may receive opposing teeth from the upper and lower jaw of the subject respectively. For example, a top notch 108 may receive a subject's top incisor and the lower notch 108 may receive the subject's lower incisor. Optionally, the notch 108 is circumferential such that opposing teeth can be seated when the subject bites down on the mouthpiece portion.

The mouthpiece portion 104 is sufficiently rigid such that seating of teeth into the notch 108 and biting down under normal pressure does not occlude the inner space between the mouthpiece portion 104 and the remainder of the body 102. Moreover, biting down and applying pressure from a tooth in a notch 108 helps secure the mouthpiece portion 104 in the subject's oral cavity in a position such that the opening is distal to the posterior teeth surfaces. Therefore, the mouthpiece portion 104 ensures positioning and secures the opening 106 in the oral cavity at least posterior to one or more of the subject's teeth. When the device is used, the mouthpiece portion 104 is positioned in the oral cavity of the subject and the subject bites down on the mouthpiece portion such that least one occlusal or incisal tooth surface seats in the notch 108.

Because the notch 108 is spaced from the opening 106, when the mouthpiece portion 104 is positioned in a subject's oral cavity such that one or more tooth surface is seated in the notch, the opening 106 is located in the oral cavity at a distance at least just posterior to the rear surface of the tooth that is seated in the notch 108. Optionally, the tooth or teeth seated in the notch 108 are incisors of the subject.

The distance the mouthpiece portion 104 extends into the oral cavity behind the tooth or teeth is a function of the length of the mouthpiece portion 104 measured between the notch 108 and the opening 106. This length (A) can vary from a minimum that causes the opening 106 to be positioned from just behind the back surface of the tooth or teeth that are seated in the notch 108 to a maximum that causes the opening to be positioned distal to the oral cavity of the subject.

Optionally, the length (A) can vary from about 1.0 centimeters (cm) to about 5.0 cm or more. By having the opening 106 positioned posterior to the tooth or teeth, when the subject exhales air into the opening during use of the device, at least a portion of the air and material located in the subject's oral cavity is excluded from the sample captured using the device 100.

For example, extension of the mouthpiece portion 104 posterior to the teeth of a subject during exhalation, reduces saliva and other mouth contents from entering the device. The extension also reduces the amount of upper airway material entering the collection device 100. Optionally, the mouthpiece portion 104 is tapered such that the opening 106 of the mouthpiece is smaller in cross sectional area than other areas of the mouthpiece portion 104.

Mouthpiece portions optionally have different sized openings to be optionally utilized for patients of different size for collecting coughs versus deep breath exhalation. A one-way valve is optionally included in the mouthpiece portion 104 to prevent backflow.

The terminal end 112 of the reservoir branch portion 110 comprises an opening 206 (shown in FIG. 2) through which exhaled air can pass. Therefore, when a subject exhales air into the opening 106 a portion of the exhaled air travels through the inner space 208 defined by the body 102 including the portion of the inner space defined by the reservoir branch portion 110.

The exhaled air continues to move through the inner space defined by the reservoir branch portion 110 and at least a portion of the air moving through the reservoir branch portion 110 inner space passes through the opening 206.

Referring again to FIG. 1, a reservoir container or balloon 114 configured to receive exhaled air passing through the branch portion's opening 206 can be positioned over the terminal end 112 of the reservoir branch portion 110. Optionally, the reservoir branch portion 110 includes a circumferential groove 204 (see FIG. 2A).

The reservoir container 114 can be secured to the reservoir branch portion 110 at the circumferential groove 204 by applying a crimping device such as a rubber band, cable or zip tie around the exterior of the reservoir container 114 over the groove 204. By tightening the crimping device, the portion of the reservoir container seated around the groove 204 can be seated into the groove 204 to secure the reservoir container 114 to the reservoir branch portion 110. The crimping device can also help seal the reservoir container 114 to the reservoir branch portion 110 such that exhaled air passing through the body 102 and through the opening 206 can enter and exit the reservoir container 114 without substantial leakage at the junction between the reservoir container 114 and the reservoir branch portion 110.

The reservoir container 114 is configured to receive at least a portion of the air exhaled into the device from a subject. Exhaled air passes through the inner space 208 of the body 102, including through the inner space of the reservoir branch portion 110. At least a portion of the air passing through the inner space of the reservoir branch portion 110 enters the reservoir container 114. Optionally, the reservoir branch portion 110 may be connected to a valve instead of a reservoir container. Optionally, the reservoir branch portion 110 has a larger inner luminal diameter or inner luminal cross-sectional area than the inner luminal diameter or cross-sectional area of the filter branch portion 120. The cross-sectional shape of the inner space of any portion of the device 100 is optionally rectangular or round.

Optionally, the reservoir container 114 is expandable upon receipt of exhaled air. Whether expandable or not, as the reservoir container receives exhaled air, increasing volume of air in the reservoir container 114 causes pressure to rise in the reservoir container 114. The rise in pressure results in increasing resistance to acceptance of exhaled air into the reservoir container 114. As the subject continues to exhale into the device, the increasing resistance of the reservoir container 114 to receipt of exhaled air eventually results in a greater portion of exhaled air being directed through the filter branch portion 120.

Differences in flow resistance allow the initial exhaled air flow to pass into the reservoir container 114 since flow will follow the path of least resistance. Air passes into the reservoir container 114 at least partially filling it. Optionally, the reservoir container 114 is made from a flexible material so that the reservoir container 114 will expand from the presence of incoming air to a maximum internal volume. The maximum internal volume is defined by the internal area of the reservoir container 114 and the elasticity of the chamber material. Optionally, the reservoir container 114 is made from material with low elasticity. A reservoir container 114 is optionally elastic to provide shock-absorbing compliance during coughing. Optionally, the reservoir container 114 is made from material with substantially no elasticity. The reservoir container 114 is optionally made from natural or synthetic materials illustratively including polyethylene, polyurethane, latex, rubber, polymeric material, and the like.

In devices with a flexible reservoir container 114, the volume of reservoir container 114 will increase to a predetermined amount with exhaled air from the subject. After at least some partial inflation of reservoir container 114, resistance to further inflation occurs providing sufficient resistance such that additional exhaled air from the lower airway passes into the filter branch portion 120.

Therefore, initially, a larger volume of exhaled air from the upper airways passes through the reservoir branch portion 110 and into the reservoir container 114 as compared to a lower volume that passes through the filter branch portion 120. For example, the diameter of the inner space defined by the reservoir branch portion 110 is optionally larger than the diameter of the inner space defined by the filter branch portion 120 causing a higher portion of initial exhaled air volume to pass into the reservoir container 114 than into the filter branch portion 120.

Once resistance to further air passage in the reservoir container 114 reaches a threshold value, an increasing percentage of additional exhaled air is directed into the filter branch portion 120. Optionally, the reservoir container 114 accepts most or all of the exhaled upper airway air and the filter branch portion 120 accepts most or all of the exhaled lower airway air. In this way, the device can selectively collect a sample from exhaled air that is less contaminated by upper airway material.

The average normal (tidal) exhalation in a healthy adult is approximately 500 ml. Of this 500 ml, the initial 150 ml of exhaled gas is from the upper airway including the trachea, and oropharyngeal space. This portion of gas is commonly referred to as the “dead space” since it does not contribute to oxygen refreshment to the lungs. The latter 350+ ml of exhaled gas comes from the lower respiratory airway and alveoli. Thus, reservoir container 114 preferably expands to 150 ml and then provides resistance to further inflation. If the reservoir container 114 is not expandable it optionally accepts 150 ml and then provides resistance to air acceptance. A greater volume or all of the lower airway breath of 350 ml is then diverted into the filter branch portion 120. By separating the initial 150 ml from the 500 ml sample, it is possible to isolate the contaminating material, such as bacteria, in the first 150 ml from the true pneumonia pathogens present the alveolar space contained in the subsequently exhaled 350 ml of air.

A reservoir container 114 is optionally of sufficient internal volume to capture all or a majority of upper respiratory air. Optionally, a reservoir container 114 has an internal volume of less than 300 ml. Optionally a reservoir container 114 has a volume between 25 ml and 300 ml. Optionally, a reservoir container 114 has an internal volume between 100 and 250 ml. Optionally, a reservoir container 114 has an internal volume of 150-225 ml. The distance between the opening 106 and the reservoir chamber 114, or the distance between the opening 106 and the reservoir branch opening 206, is optionally 50 cm or less.

The reservoir container 114 is optionally adjusted or replaced with a different size chamber to collect different volumes depending on the size of the patient. For instance, a child may use a chamber that collects a volume of only 50 ml, while a large adult may use a collection volume of 300 ml.

The filter branch portion 120 directs exhaled air to a filter 202 (See FIG. 2B) for collection of sample. The terminal opening 106 of the mouthpiece portion 104 has a central axis, which is optionally in alignment with the long axis of the conduit, for example as shown in FIG. 2A. The filter is spaced from the terminal opening and in communication with the inner space and the filter is positioned at a predetermined angle relative to the central axis of the terminal opening. The predetermined angle is less than ninety degrees. At least a portion of the exhaled air passes through the filter. The filter 202 is optionally less than ninety degrees and in substantial alignment with the opening 106 of the mouthpiece portion 104. Substantial alignment means that there is a direct linear path between the opening 106 and the filter. The filter 202 can be secured against the filter branch portion 120 with a cap 116. The volume of the inner space 208 between the opening 106 and the collecting filter 202 can optionally be 500 ml of less.

The filter 202 optionally comprises any suitable material to allow gaseous fluid (e.g. exhaled air) to pass through the filter while trapping materials contained in the fluid such as pathogens including virus particles, bacteria, yeast, fungus, and the like. A filter optionally traps environmental contaminants such as asbestos, fiberglass, pesticides and the like. A filter optionally traps cells or cellular material, including, for example, nucleic acid. A filter is optionally made from paper, gelatin, polymers, glass, or other material. The material is optionally operable for collecting target material.

Optionally a filter is sized to collect some targeted materials while letting others pass thorough. Illustratively, a filter is used to trap bacteria. Optionally bacteria are S. pneumonia. Optionally, a filter traps virus particles. Optionally, a filter is made of reactive material that will interact with a target molecule or pathogen thereby trapping the molecule or pathogen in the filter while selectively allowing non-targets to pass through the filter. The filter is optionally less than 100% effective at trapping a target. Example filter materials suitable for use with the device are known in the art and are obtained from sources known in the art, illustratively, 3M Corp. (St. Paul, Minn.). Optionally, VBMax® filters from A-M Systems, Inc. (Carlsborg, Wash.) are used.

Optionally, the filter is present in a cartridge that attaches to the filter branch portion 120. Optionally, a filter cartridge attaches to the filter branch portion 120 by way of a press fit. Optionally, a filter cartridge attaches to the filter branch portion by a screw fit, clamp, bayonet, intermediate connector, or other type of fitting known in the art.

Optionally, the filter may be replaced by one or more detectors. A detector is optionally positioned in communication with air exhaled through the filter branch portion 120 so that lower airway/alveolar air is exposed to a detector during an exhalation of a subject. Optionally, a second detector is present within the filter branch portion 120.

A detector is optionally operable to detect the presence or absence of a bacteria, virus, fungus, antibody, protein, or chemical such as carbon dioxide, nitric oxide, alcohol and the like. A detector is optionally capable of detecting the presence of alcohol in the lower or upper airway of a subject. A detector is optionally a surface, a labyrinth, a trap, sticky substance, warmed or cold surface, metal, or electrical, chemical or mechanical sensor. A sensor or other detector is illustratively operable to detect a molecule or pathogen, either by color, optical, electronic or other chemical means.

FIG. 2A is an exploded side-on view of the device 100 showing the terminal end of the filter branch portion 120, filter 202, and the cap 116. One or more opening in the cap 116 allows for passage of air through the filter 202 and out of the device 100.

FIG. 2C is a side-on midline longitudinal cross sectional view of the device 100. The arrows schematically denote example flow paths for exhaled air thought the body's inner space. For example, exhaled air enters the conduit 103 through the opening 106 and can proceed through the reservoir branch portion 110 and through the opening 206. The exhaled air can also proceed through the conduit 103 including the filter branch portion 120.

As the air passes through the terminal opening 210 of the filter branch portion 120 it may penetrate the filter 202 and exit the device through an opening in the cap 116. Optionally, the cap 116 is attached to the filter branch portion 120 using a threaded interlocking system in which threads 212 on the cap mate with complementary threads on the filter branch portion 120. The cap 116 can also be secured to the filter branch portion 120 using a variety of mechanisms. For example, in addition to a threaded interlocking system, the cap 116 can be press-fit onto the filter branch system.

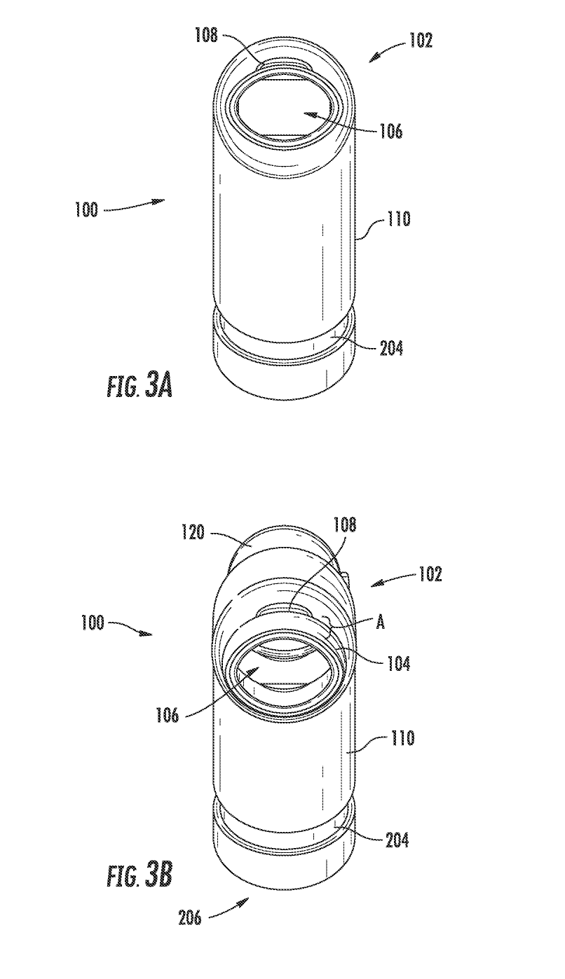

Referring now to FIGS. 3A and 3B, the device 100 is illustrated as viewed from the mouthpiece portion 106. The opening 106 optionally has a narrow cross sectional region and a wide cross sectional region. For example, the opening 106 may be oval, but many alternative shapes could be used as described above. FIG. 3B shows a perspective view the device 100 illustrated towards the mouthpiece portion 104. In this example, a notch 108 is shown into which a subject's teeth are positioned when the mouthpiece portion is positioned within the oral cavity of a subject. When inserted into the oral cavity as shown, the subject optionally bites down on the device such that its superior incisors are seated in the notch 108, and optionally, such that the subject's inferior incisors are positioned in a similar notch on the opposite side of the outer surface of the mouthpiece portion 104. The notch 108 is separated from the opening 106 by a distance (A) such that when a tooth is positioned in a notch 108, the opening extends behind the teeth further into the oral cavity.

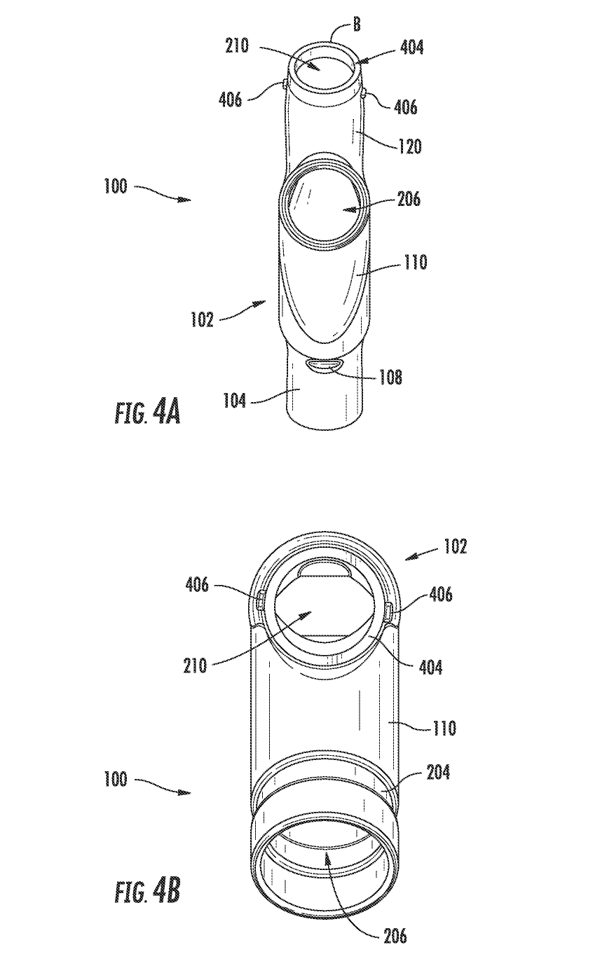

FIG. 4A illustrates the device 100 as viewed from the reservoir branch portion 110. In this view, the terminal end of the filter branch opening 210 is illustrated. The terminal end surrounds the opening 210 and includes a terminal planar surface 404 with a width (B). The filter 202 can be positioned against the terminal surface 404 and the filter can be sandwiched between the terminal surface 404 and a portion of the cap 116 to secure the filter 202 in position across the opening 210.

FIG. 4B illustrates the device 100 as viewed from the filter branch portion 120 without the cap 116. In this view, the terminal surface 404 is illustrated and two locking protrusions 406 are shown. The protrusions 406 can be mated with grooves in the cap 116 to secure the cap in position on the filter branch portion 120. It is understood, that alternative mechanisms for attaching caps can be used. Moreover, in some examples a cap is not used. For example, the filter could be positioned in a slot that locates the filter in the path of the air flow through the conduit.

FIG. 5 illustrates the device 100 as viewed from the filter branch portion 120 with the cap 116 in position. The cap 116 is positioned over the filter branch opening 210. In use, a filter 202 may be positioned under the cap 116 such that it is held between the terminal end of the filter branch portion 120 and the cap 116. As described above, exhaled air passes through the inner space of the body 102 and passes through the filter 202. To allow passage of air through the filter when the cap 116 is positioned as shown, the cap 116 includes one or more cap opening 504. In the illustrated example, the cap openings 504 are framed by one or more rigid or semi-rigid ribs 502.

At least a portion of the exhaled air can pass through the filter 202 and exit the device through a cap opening 504. The ribs 502 are optionally rigid enough to help hold the filter in position under the pressure of the air exhaled through the filter 202. Subsequent to collection of a sample, e.g., subsequent to one or more exhalation events (e.g. one or more cough or other exhalation) from the subject, the cap 116 can be optionally removed. Once the cap 116 is removed the filter may optionally be removed for further analysis. The filter can also be subjected to further analysis without being removing the cap 116 and/or the filter 202.

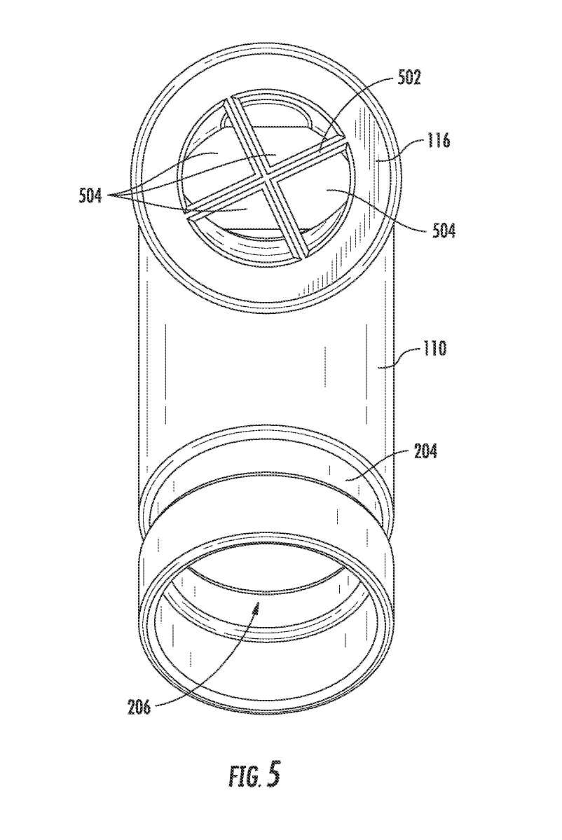

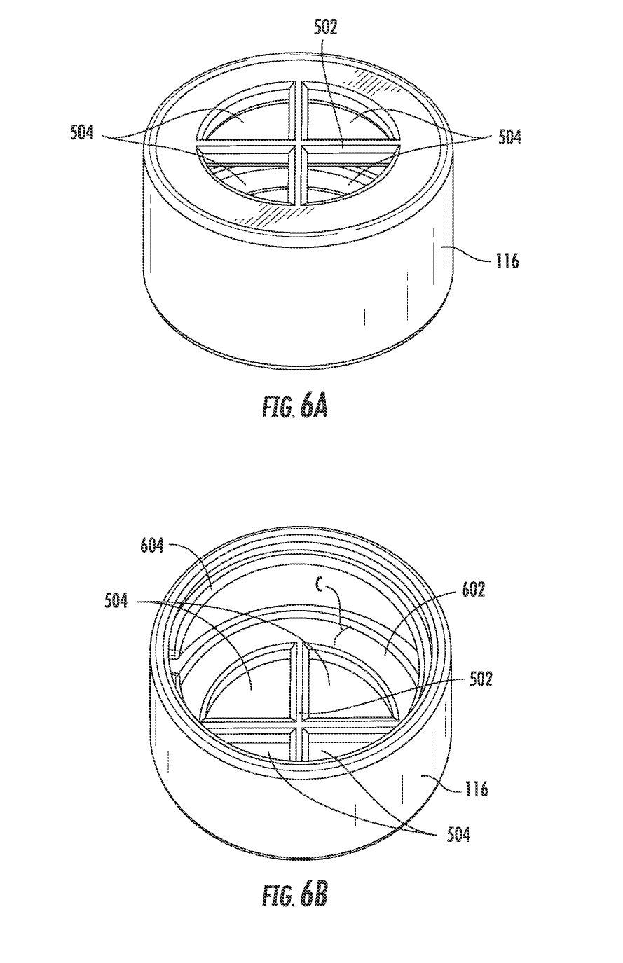

FIGS. 6A and 6B are schematic perspective views of an example cap 116. The example cap 116 includes four cap openings 504 and four ribs 502. The ribs 502 meet at a central point, which is positioned over the center of the opening 210 and the filter 202 when the filter is positioned over the opening 210.

As shown in FIG. 6B, the cap 116 includes an inner rim 602 encircles the cap openings 504. The inner rim 602 has a width (C). A portion of the filter 202 can optionally be sandwiched between the inner rim 602 and the terminal surface 404 of the filter branch portion 120. Thus, the filter 202 can be held in place at its periphery by the inner rim 602 and terminal surface 404 and the more central portions of the filter can be supported against the pressure of exhaled air by one or more cap ribs 502. FIG. 6B also illustrates threads 604 for securing the cap 116 to the filter branch portion 120.

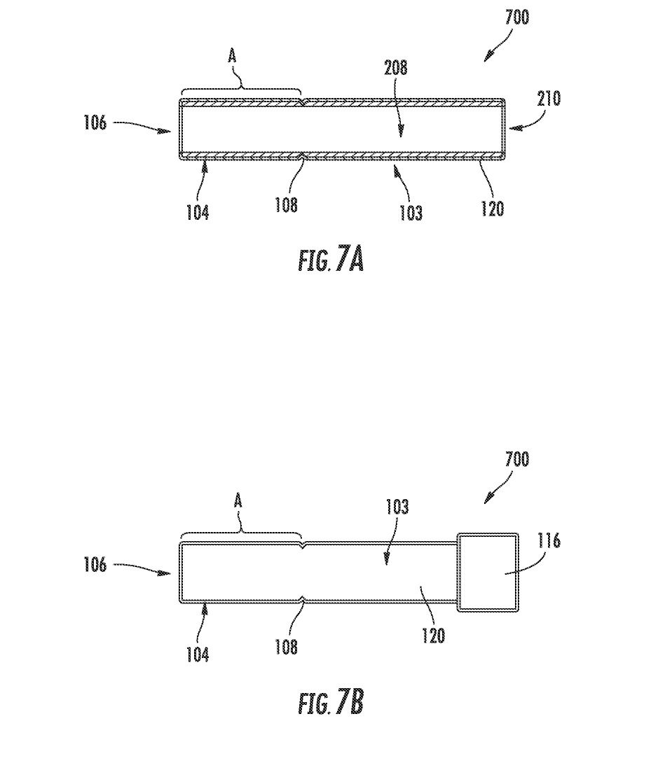

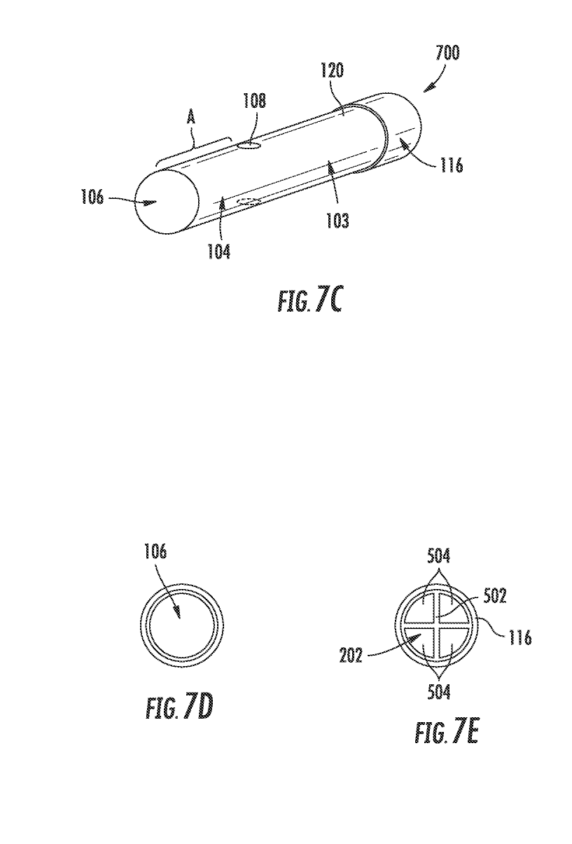

FIGS. 7A-7E illustrate an example aerosol collection device 700. The device 700 includes a body comprising a conduit 103 which defines an inner space or lumen 208. The conduit 103 includes a mouthpiece portion 104 and a filter branch portion 120. At least a portion of the mouthpiece portion 104 is inserted into the oral cavity of a subject for use and the filter branch portion 120 extends away from the oral cavity and can direct exhaled air through a filter 202.

As shown, the conduit 103, including the mouthpiece portion 104 and the filter branch portion 120, can be a straight tubular structure with an opening 106 defined by the mouthpiece portion 106 and an opening defined by the filter branch portion 210.

Optionally, at least one indention or notch 108 is located in the mouthpiece portion at a fixed distance (A) from the opening 106. One or more of the subject's teeth can be located in a notch during use. By biting down into the notch 108, the opening 106 is positioned behind the teeth (e.g. towards the larynx) in the oral cavity. The subject exhales into the opening 106 and air moves into the opening, through the inner space 208 or lumen, and through the opening 210.

As described in regard to the device 100, a filter 202 can be positioned distal to the opening 210, which can be held in place by a cap 116. Exhaled air passes through the opening, through the filter 202, and through at least one cap opening 504. Sample material is collected on the filter 202 as air passes through the same and the cap 116 and filter 202 are optionally removed for analysis. The cap 116 and/or filter 202 can also be left in attachment to the filter branch portion for analysis of the sample collected on the filter 202.

The device 700 excludes at least a portion of upper airway contaminants from the collected sample. For example, as described above in relation to the example device 100, by positioning the opening 106 behind the teeth a distance into the oral cavity, contamination from the oral cavity can be reduced. Thus, the collected sample can comprise a higher proportion of lower air way material, including pathogens.

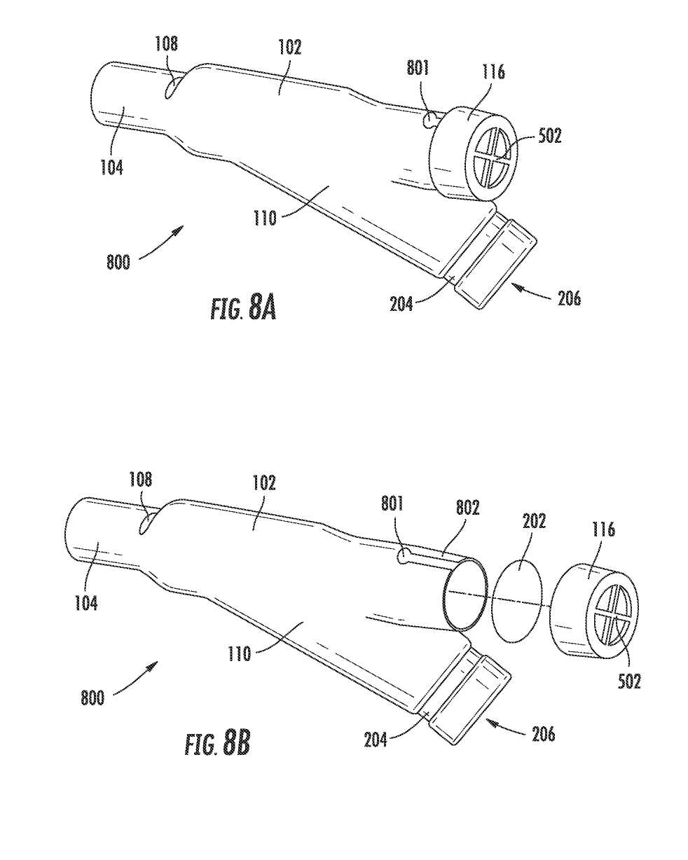

FIGS. 8A and 8B illustrate an example aerosol collection device 800. The device 800 can be used with rapid immunoassay techniques to detect the presence of antigens in a sample collected with the device. The device 800 includes a receptacle 801 and a groove 802. Optionally, the groove is approximately 0.5 mm deep. Buffered liquid solution containing antibody specific for a target antigen is deposited in the receptacle 801 and can flow by gravity along the groove until it contacts the filter 202. The filter 202 can comprise antigen collected from the subject's respiratory tract and can also comprise target antigen for use as control indicator.

Once the solution contacts the filter 202, it flows by gravity or surface tension along the surface of the filter 202 to detect the presence of antigens on the substrate using a lateral flow assay provided directly on the filter 202.

Optionally, conjugated antibody-antigens collect in a first zone, zone 1, with subsequent color change if antigen is present. The solution then optionally flows through a second zone, zone 2, with non-collected deposited antigen to demonstrate color change as a control.

The antibody-antigen pairs are used to produce a measurable signal in response to binding. The signal or label is accomplished in a variety of ways. For example, binding can be detected using conjugation to a fluorescent or colored molecule, change in electro-active groups, magnetic particles, or radioactive elements. Optionally, the antibodies used are linked to an enzyme to create a color change, commonly known as an ELISA assay. Optionally, a separation step or multiple steps for purification or concentration of the antigen or antibody-antigen conjugate is used. The antibody used are optionally polyvalent or monovalvent. The assay is optionally qualitative for comparison with a calibrator or quantitative with additional calibration. The assay type is optionally competitive or non-competitive.

Because the filter trapped material from lower airway exhaled air, the antibody can contact material from the subject's lower airway air that is located on the filter. For example, an antibody specific for material in the collected sample can contact bacteria, bacterial antigens, virus or viral antigens, cells, cell antigens, or other target materials to be detected on the filter. Example antigens may be polysaccharides or proteins from bacteria such as Streptococcus pneumoniae, Mycobacterium tuberculosis, haemophillus, influenza, or Respiratory Syncytial Virus.

The system of placement of zones of reagents, antibodies, and/or detectors on the filter can be used to eliminate use of separate extraction and transfer of antigens with resultant loss of sensitivity.

The described devices can be used to collect a sample from exhaled lower respiratory tract air. The described devices can also be used as a diagnostic aid for the detection and diagnosis of disease or abnormality. Because the devices are able to distinguish between the contents of the lower respiratory tract and the upper respiratory tract, the devices are operable for detecting the presence of alcohol in the blood independent of recent consumption and contamination by residuary alcohol in the mouth or upper digestive tract.

Optionally, the devices can be used for the diagnosis of infectious pneumonia. The devices segregate lower respiratory gas and aerosolized material from contaminants such as liquid from the mouth and gas or aerosolized pathogens from the oral cavity. Illustratively, a filter 202 traps the lung pathogens without a complicating second chamber. Optionally, a fluid dynamics resistance pressure system maintains segregation of gases during exhalation. Optionally, a shaped mouthpiece effectively prevents large amounts of oral liquids from entering the device. Optionally, an air-liquid trap uses gas diversion to further reduce liquid contamination. Optionally, a deformable reservoir chamber of variable volume is adjusted to the lung volume of individual patients such as a child with reduced lung volume or an adult with higher lung volume.

Pathogens detectable or collectable by the described devices optionally include, but are not limited to, bacteria, including, Streptococcus pneumonia, H. influenza, M. Tuberculosis, Staphylococcus aureus, Gram-negative bacilli, Legionella species, M. pneumoniae, C. pneumoniae, and viruses, and fungi. For example, optional pathogens detected are pneumocystis, jroveci fungus, anthrax fungus, H1N1 virus. Analytes such as chemicals detectable or collectible by the described devices illustratively include proteins such as amylase, nucleic acids, nitric oxide, carbon dioxide, acetone, alcohol including ethanol, and surfactant. The amount of alcohol and acetone in a sample of alveolar air will allow computation of total body fat as described in U.S. Pat. No. 4,114,422, the contents of which are incorporated herein by reference.

The described devices have a number of clinical uses in interrogating the respiratory system. For example, nucleic acid, such as DNA, RNA or fragments thereof from pathogens causing pneumonia of the lower airway can be sampled using the devices. Exhaled ethanol from the bloodstream is optionally sampled using the devices. Samples of asbestos or coal in the lung are also optionally sampled to differentiate upper and lower airway origins. Cancer cells or pre-cancerous cells from the lung are optionally collected. Cancer cells or pre-cancerous cells from the lower airway are differentiable from such cells present in upper airway origins. Oxygen in gas may also be measured separately from oxygen in the liquid material in the upper airway.

The devices can be used to collect samples of alveolar aerosols. Alveolar aerosols are at least partially separated from upper airway air by passing the alveolar air through a filter 202 not exposed to upper airway air and collecting a sample from the filter 202.

Optionally, a sample is collected from a second breath of a subject by passing alveolar aerosols from the second breath through the filter 202. Optionally, alveolar aerosols from multiple breaths are passed through the filter. Optionally, upper airway air that is separated from lower alveolar air is collected and/or sampled.

The disclosed devices can be used to detect and/or diagnose a pathogen or disease. A disease is optionally an infectious disease or a non-infectious disease such as cancer. Alveolar aerosols are collected from lower airway air and passed through a filter 202 not exposed, or with reduced exposure, to upper airway air. Collected material is used to detect the presence or absence of a pathogen in the alveolar air and the upper airway air to diagnose the presence of a disease in the subject. Collected material can also be used to detect the presence or absence of a cell, including cancerous cells or pre-cancerous cells, in the alveolar air and the upper airway air to diagnose the presence of a disease in the subject.

The disclosed devices can be used to detect the presence and/or concentration of a volatile analyte in the subject's bloodstream. Alveolar air is separated, at least partially, from upper airway air and the separated air is exposed to a detector to detect the presence or absence of an analyte in the alveolar air. Optionally, upper airway air is exposed to a second detector and the presence or absence of an analyte in the upper airway air is detected.

Many modifications and other embodiments of the devices, methods and kits set forth herein will come to mind to one skilled in the art to which these devices, methods and kits pertains having the benefit of the teachings presented in the foregoing description. Therefore, it is to be understood that the devices, methods and kits are not to be limited to the specific embodiments disclosed and that modifications and other embodiments are intended to be included within the scope of the appended claims. Although specific terms are employed herein, they are used in a generic and descriptive sense only and not for purposes of limitation.

1. A device for collecting an aerosol sample from a subject, comprising:
a. a conduit that defines an inner space for the passage of exhaled air, wherein the conduit has a long axis and a first opening for receiving exhaled air from the subject; and
b. a filter for collecting a sample from the exhaled air, wherein the filter is in communication with the inner space and is substantially aligned with the first opening.

2. The device of claim 1, wherein the cross-sectional area of the inner space along the conduit taken perpendicular to the long axis is constant between the opening and the filter.

3. The device of claim 1, wherein the a portion of the conduit is configured for placement in the oral cavity of the subject, and wherein the conduit comprises at least one notch spaced from the first opening, said notch configured to receive a tooth surface of the subject when the portion configured for placement in the oral cavity is operably located in the oral cavity.

4. The device of claim 3, wherein the distance between the notch and the first opening measured along the body's outer surface is about 1.0 cm or greater.

5. (canceled)

6. (canceled)

7. The device of claim 1, further comprising a branch segment defining an inner space in communication with inner space of the conduit that extends from the conduit at an angle relative to the long axis of the conduit.

8. The device of claim 7, wherein the branch segment terminates in a reservoir configured to receive exhaled air.

9. The device of claim 8, wherein the opening to the branch segment from the conduit has a larger cross-sectional area than a cross sectional area of the conduit taken perpendicular to the long axis.

10. The device of claim 8, wherein the reservoir has an inner volume less than 300 ml.

11. The device of claim 8, wherein the reservoir expands upon filling with exhaled air from the subject.

12. The device of claim 11, wherein pressure in the expanded reservoir directs exhaled air along the conduit and through the filter.

13. The device of claim 8, wherein the exhaled air received in the reservoir increases pressure in the reservoir causing the direction of additional exhaled air along the conduit and through the filter.

14. The device of claim 1, wherein the filter comprises nucleic acid or an antigen from a lung pathogen.

15. (canceled)

16. The device of claim 14, wherein at least a portion of the lung pathogen is collected from the exhaled air.

17. The device of claim 14, further comprising a solution containing an antibody specific for the antigen.

18. The device of claim 17, wherein the conduit further comprises a reservoir configured to receive the solution and channel configured to deliver at least a portion of the received solution into contact with the filter.

19. The device of claim 14, wherein the lung pathogen is selected from the group consisting of streptococcus pneumoniae, mycoplasma tuberculosis, Chlamydia pneumoniae, legionella sp, influenza viruses, respiratory syncytial virus, parainfluenza, adenovirus, rhinovirus, human bocavirus, influenza, Mycoplasma pneumoniae, hantavirus, pneumocystis, jroveci fungus, anthrax fungus, H1N1 virus, and cytomegalovirus.

20. (canceled)

21. (canceled)

22. A device for collection of an aerosol sample from a subject, comprising:
a. a body defining a inner space and having a segment with a substantially linear long axis, the segment comprising a first portion configured for insertion into the oral cavity of a subject, said first portion comprising a terminal opening through which air exhaled from the subject enters the inner space;
b. a filter spaced from the terminal opening and in communication with the inner space, wherein the filter is substantially aligned with the long axis of the segment and wherein at least a portion of the exhaled air passes through the filter.

23-40. (canceled)

41. A method of collecting an aerosol sample from a subject, comprising:
a. providing a conduit that defines an inner space and that has a first opening for receiving exhaled air from the subject;
b. receiving exhaled air from the subject into the inner space through the first opening; and
c. positioning a filter in communication with the inner space, wherein the filter is substantially aligned with the first opening.

42. The method of claim 41, wherein the exhaled air passes through the filter.

43. The method of claim 42, wherein the filter captures a sample from the exhaled air.

44. (canceled)

45. (canceled)
